(19) United States
(12) Patent Application Publication
(10) Pub. No.: US 2025/0082209 A1
(43) Pub. Date: Mar. 13, 2025

(54) BIOLOGICAL INFORMATION MEASUREMENT DEVICE

(71) Applicant: OMRON HEALTHCARE Co., Ltd., Kyoto (JP)

(72) Inventors: Kosuke INOUE, Kyoto (JP); Kengo NISHIYAMA, Kyoto (JP); Masahiko YUMOTO, Kyoto (JP); Yuka TANABE, Kyoto (JP); Minoru FUJINO, Kyoto (JP); Asa HIRASAWA, Kyoto (JP); Takahiro HAMAGUCHI, Kyoto (JP); Yoshikazu INAMI, Kyoto (JP); Rika KATO, Kyoto (JP); Hiroyuki KANDA, Kyoto (JP); Yuta KUDO, Kyoto (JP); Masaaki NONOYAMA, Kyoto (JP); Yuki SUZUKI, Kyoto (JP); Yuki KIMPARA, Kyoto (JP); Haruna MIYAZAKI, Kyoto (JP); Susumu HONDA, Kyoto (JP); Masato KOYAMA, Tokyo (JP)

(21) Appl. No.: 18/956,179

(22) Filed: Nov. 22, 2024

Related U.S. Application Data
(63) Continuation of application No. PCT/JP2023/004656, filed on Feb. 10, 2023.

(30) Foreign Application Priority Data
Jul. 7, 2022 (JP) 2022-109760

(51) Int. Cl. A61B 5/02; A61B 5/00

(52) U.S. Cl. CPC A61B 5/02028; A61B 5/7415; A61B 5/742

(57) ABSTRACT

A biological information measurement device includes a pulse acquisition mechanism configured to detect a pulse of a human body, a pulse interval calculation mechanism configured to calculate, based on the pulse, a pulse interval between one beat and a beat immediately before the one beat, and a display mechanism configured to display a level indicator visually indicating at least one of the pulse interval or an amount of change from another pulse interval immediately before the pulse interval. The level indicator indicates, per beat of the pulse detected, the pulse interval or the amount of change.

### CROSS-REFERENCE TO RELATED APPLICATIONS

This application is the U.S. national stage application filed pursuant to 35 U.S.C. 365 (c) and 120 as a continuation of International Patent Application No. PCT/JP2023/004656, filed Feb. 10, 2023, which application claims priority to Japanese Patent Application No. 2022-109760, filed Jul. 7, 2022, which applications are incorporated herein by reference in their entireties.

### TECHNICAL FIELD

The present invention relates to a biological information measurement device for measuring a pulse of a living body, and more particularly to a biological information measurement device for providing information related to an interval of a measured pulse.

### BACKGROUND ART

In recent years, it has become widespread to perform health management by measuring information related to the body and health of an individual such as a blood pressure value with a measurement device and recording and analyzing the measurement result. In particular, arrhythmia such as atrial fibrillation (AF) may lead to cerebral and cardiovascular diseases. Thus, it is effective to detect a fluctuation in a pulse interval with the above-described device and notify a user so that the user can easily recognize the fluctuation.

In the related art, it has been known to provide information related to such a pulse interval based on biological information acquired when blood pressure measurement is performed using a blood pressure monitor. For example, Patent Document 1 discloses a blood pressure monitor capable of storing a pulse wave to be used for measurement of a blood pressure value and displaying a pulse wave graph simultaneously with the blood pressure value. Patent Document 1 also discloses that a heart mark displayed on a screen blinks in accordance with pulsation during blood pressure value calculation.

According to the blood pressure monitor described in Patent Document 1, a user can recognize a pulse interval by checking a blinking interval of the heart mark blinking in accordance with pulsation during blood pressure measurement. Since a time-series graph of a signal level of a pulse wave is subsequently displayed as a time-series pulse wave graph, it is possible to check a pulse interval (and a fluctuation thereof) by reading such a graph.

### CITATION LIST

### Patent Literature

Patent Document 1: JP 2007-98003 A

### SUMMARY OF INVENTION

### Technical Problem

However, only blinking the heart mark in accordance with the pulsation as in the technique described in Patent Document 1 is not enough to easily recognize a fluctuation in a pulse interval, and there is a problem in that an important fluctuation in a pulse interval is overlooked. Even when the time-series graph of the signal level of the pulse wave is subsequently displayed, it is difficult for a typical user who has no medical knowledge to correctly read information of arrhythmia from the time-series graph. In addition, even when the user can read the information of arrhythmia from the time-series graph, the user cannot intuitively and sensuously recognize arrhythmia, but only recognizes the information as objective unrealistic information, and may not have a sense of urgency.

The present invention has been made in view of the above circumstances, and an object thereof is to provide a technique that can enable a user who has no medical knowledge to easily recognize arrhythmia using a measurement device capable of detecting a pulse and enhance the user's recognition about arrhythmia.

### Solution to Problem

The present invention adopts the following configurations to solve the above-described problems. That is, a biological information measurement device includes a pulse acquisition mechanism configured to detect a pulse of a human body, a pulse interval calculation mechanism configured to calculate, based on the pulse, a pulse interval between one beat and an immediately preceding beat, and a display mechanism configured to display a level indicator visually indicating at least one of the pulse interval or an amount of change from another pulse interval immediately before the pulse interval. The level indicator indicates, per beat of the pulse detected, the pulse interval or the amount of change.

The level indicator described herein is only required to indicate a predetermined feature amount by using a non-numerical display (for example, the size of a display region), and the shape and display mode thereof are not limited. For example, the level indicator may visually indicate the pulse interval or the amount of change by using at least one of a length, an area, or an angle of a region in which display is activated at the display mechanism or the number of the regions. Such a configuration allows the pulse interval or the amount of change from another pulse interval immediately before the pulse interval for each beat to be easily recognized. Thus, even a user who has no medical knowledge can sensuously recognize the degree of change in the pulse interval for each beat and intuitively feel a sense of discomfort particularly when there is an abnormality.

The level indicator may visually indicate the pulse interval or the amount of change by using a size indicated by a display region in which display is activated in the level indicator. The level indicator may be composed of a plurality of display segments and represent the size of the display region depending on whether the number of the plurality of display segments in which display is activated is large or small.

The expression “display is activated” used here refers to a transition to a display state in a region that can be switched between a display state and a non-display state (that is, deactivation of display refers to a transition to a non-display state). For example, when the display mechanism is an LCD, the expression “display is activated” refers to display output in a display region on the display, and when the display mechanism is an LED light or the like, this expression refers to the light being turned on. Each display segment is a unit of a display region that can be individually switched between a display activation state and a display deactivation state, and a shape thereof is not particularly limited.

Such a configuration allows the difference in the pulse interval or the amount of change thereof to be indicated by the size of the display region, allowing the user to easily recognize the degree of change in the pulse interval.

The level indicator may maintain activation of display of a peak level portion of one pulse interval or one amount of change from display of the one pulse interval or the one amount of change until display of a next pulse interval or a next amount of change.

Such a configuration allows the peak level portion indicating the previous pulse interval or the amount of change thereof to be displayed until immediately before the next pulse interval or the amount of change thereof is displayed, allowing the user to more clearly recognize the degree of change in the pulse interval.

The display mechanism may further display a sub-indicator including a plurality of sub-display segments corresponding one-to-one to the plurality of display segments of the level indicator. The level indicator may maintain activation of display of a peak level portion of one pulse interval or one amount of change from display of the one pulse interval or the one amount of change until display of a next pulse interval or a next amount of change. The sub-indicator may activate, per beat, display of a sub-display segment of the plurality of sub-display segments that corresponds to the display of the peak level portion of the pulse interval or the amount of change indicated per beat by the level indicator and maintain display of each sub-display segment activated until the detection by the pulse acquisition mechanism is finished.

In such a display mode, it is possible to easily recognize the degree of variation in the pulse interval. That is, when the blood pressure measurement by the blood pressure measurement device has been finished, that is, when the pulse acquisition by the blood pressure measurement device has been finished, the larger the number of the sub-display segments in which the display is activated in the sub-indicator is and the wider the range of the displayed positions is, the larger the variation in the pulse interval is. When atrial fibrillation occurs during the blood pressure measurement, the variation in the pulse interval becomes large, and thus the user can sensuously recognize whether or not there is a risk of atrial fibrillation by checking a mode of display activation of the sub-display segments of the sub-indicator.

The level indicator may be composed of a plurality of display segments, a display segment in which display is activated in the level indicator may make a transition, and the pulse interval or the amount of change may be indicated by a length of a distance of the transition.

Such a configuration allows the difference in the pulse interval or the amount of change thereof to be indicated by the length of the distance of the transition of the display segment in which the display is activated, allowing the user to easily recognize the degree of change in the pulse interval.

The level indicator may represent, per transition of the display segment indicating one pulse interval or one amount of change, a length of a distance of the transition by highlighting the display segment activated at a terminal end of the distance of the transition. Such a configuration allows the user to more clearly recognize the length of the distance of the transition.

The level indicator may maintain, until display of the display segment indicating the terminal end of the transition of the display segment indicating the one pulse interval or the one amount of change is activated, activation of the display segment indicating the terminal end of the transition related to at least an immediately preceding pulse interval or an immediately preceding amount of change. Such a configuration allows the terminal end of the transition distance indicating the previous pulse interval or the previous amount of change and the terminal end of the transition distance indicating the next pulse interval or the next amount of change to be clearly compared with each other, allowing the user to more easily recognize the degree of change in the pulse interval.

The level indicator may have an entire display region configured in a ring shape, the display segment in which the display is activated in the level indicator may repeat, per beat, a transition in a fixed direction, and the pulse interval may be indicated by the length of the distance of the transition.

The level indicator may have an entire display region configured in a band shape extending in a left-right direction, the display segment in which the display is activated in the level indicator may repeat, per beat, a transition in a fixed direction of the left-right direction, and the pulse interval may be indicated by the length of the distance of the transition. The level indicator may have an entire display region configured in a band shape extending in an up-down direction, the display segment in which the display is activated in the level indicator may repeat, per beat, a transition in a fixed direction of the up-down direction, and the pulse interval may be indicated by the length of the distance of the transition.

The level indicator may have an entire display region configured in a shape including at least part of a circumference and a pointer extending from an inside of the circumference toward the circumference and visually indicate the pulse interval or the amount of change by using a position on the circumference, the position being indicated by the pointer.

The level indicator may indicate the pulse interval or the amount of change in synchronization with a waveform of the pulse detected. According to this, since the display is performed in time with the actual pulsation, the user can recognize the degree of change in the pulse interval with more physical sensation, and when there is a pulse abnormality, the user can recognize the abnormality more easily.

The length, the area, or the angle of the region in which the display is activated or the number of the regions in the level indicator linearly may change in accordance with the pulse interval or the amount of change. Since the region or regions in which the display is activated in the level indicator change in proportion to the pulse interval or the amount of change, the user can intuitively and visually recognize a fluctuation in the pulse interval or the amount of change thereof.

The length, the area, or the angle of the region in which the display is activated or the number of the regions in the level indicator may change in a monotonically increasing non-linear manner in accordance with the pulse interval or the amount of change. According to this, it is possible to suppress a variation due to the difference in the magnitude of the pulse rate and to visually indicate the pulse interval or the amount of change by appropriately changing the display.

The biological information measurement device may further include a sound output mechanism configured to output, per beat of the pulse detected, a sound indicating the pulse interval or the amount of change in synchronization with display of the level indicator. The sound output mechanism may indicate a difference in the pulse interval or the amount of change depending on a difference in a pitch of an output sound.

With such a configuration, the user can recognize the pulse interval or the amount of change thereof not only visually but also auditorily and can thus recognize the degree of change in the pulse interval more clearly.

The pulse interval calculation mechanism may perform a predetermined calculation on the calculated pulse interval or the amount of change obtained by using the pulse interval, and the region in which the display is activated in the level indicator may be determined based on a value calculated through the calculation.

The configurations and processing described above can be combined with one another to constitute the present invention unless the combination leads to contradiction.

### Advantageous Effects of Invention

According to the present invention, it is possible to provide a technique that can enable a user who has no medical knowledge to easily recognize arrhythmia using a measurement device capable of detecting a pulse and enhance the user's recognition about arrhythmia.

### BRIEF DESCRIPTION OF DRAWINGS

FIG. 1 is a schematic diagram illustrating an outline of a device configuration and a functional configuration of a blood pressure measurement device according to Example 1.

FIG. 2(A) is a first diagram illustrating an example of a level indicator displayed at an image display mechanism of the blood pressure measurement device according to Example 1.

FIG. 2(B) is a second diagram illustrating an example of the level indicator displayed at the image display mechanism of the blood pressure measurement device according to Example 1.

FIG. 2(C) is a third diagram illustrating an example of the level indicator displayed at the image display mechanism of the blood pressure measurement device according to Example 1.

FIG. 3 is an explanatory diagram for describing a pulse wave signal detected during blood pressure measurement and a time.

FIG. 4(A) is a first diagram illustrating a variation of the level indicator displayed at the image display mechanism of the blood pressure measurement device.

FIG. 4(B) is a second diagram illustrating a variation of the level indicator displayed at the image display mechanism of the blood pressure measurement device.

FIG. 4(C) is a third diagram illustrating a variation of the level indicator displayed at the image display mechanism of the blood pressure measurement device.

FIG. 5(A) is a fourth diagram illustrating a variation of the level indicator displayed at the image display mechanism of the blood pressure measurement device.

FIG. 5(B) is a fifth diagram illustrating a variation of the level indicator displayed at the image display mechanism of the blood pressure measurement device.

FIG. 5(C) is a sixth diagram illustrating a variation of the level indicator displayed at the image display mechanism of the blood pressure measurement device.

FIG. 6(A) is a seventh diagram illustrating a variation of the level indicator displayed at the image display mechanism of the blood pressure measurement device.

FIG. 6(B) is an eighth diagram illustrating a variation of the level indicator displayed at the image display mechanism of the blood pressure measurement device.

FIG. 6(C) is a ninth diagram illustrating a variation of the level indicator displayed at the image display mechanism of the blood pressure measurement device.

FIG. 7(A) is a tenth diagram illustrating a variation of the level indicator displayed at the image display mechanism of the blood pressure measurement device.

FIG. 7(B) is an eleventh diagram illustrating a variation of the level indicator displayed at the image display mechanism of the blood pressure measurement device.

FIG. 7(C) is a twelfth diagram illustrating a variation of the level indicator displayed at the image display mechanism of the blood pressure measurement device.

FIG. 8(A) is a thirteenth diagram illustrating a variation of the level indicator displayed at the image display mechanism of the blood pressure measurement device.

FIG. 8(B) is a fourteenth diagram illustrating a variation of the level indicator displayed at the image display mechanism of the blood pressure measurement device.

FIG. 8(C) is a fifteenth diagram illustrating a variation of the level indicator displayed at the image display mechanism of the blood pressure measurement device.

FIG. 8(D) is a sixteenth diagram illustrating a variation of the level indicator displayed at the image display mechanism of the blood pressure measurement device.

FIG. 9(A) is a seventeenth diagram illustrating a variation of the level indicator displayed at the image display mechanism of the blood pressure measurement device.

FIG. 9(B) is an eighteenth diagram illustrating a variation of the level indicator displayed at the image display mechanism of the blood pressure measurement device.

FIG. 9(C) is a nineteenth diagram illustrating a variation of the level indicator displayed at the image display mechanism of the blood pressure measurement device.

FIG. 10 is a schematic diagram illustrating an outline of a device configuration and a functional configuration of a blood pressure measurement device according to Example 2.

FIG. 11(A) is a first diagram illustrating an example of a level indicator displayed at an image display mechanism of the blood pressure measurement device according to Example 2.

FIG. 11(B) is a second diagram illustrating an example of the level indicator displayed at the image display mechanism of the blood pressure measurement device according to Example 2.

FIG. 11(C) is a third diagram illustrating an example of the level indicator displayed at the image display mechanism of the blood pressure measurement device according to Example 2.

FIG. 12(A) is a twentieth diagram illustrating a variation of the level indicator displayed at the image display mechanism of the blood pressure measurement device according to Example 1.

FIG. 12(B) is a diagram illustrating a variation of the level indicator displayed at the image display mechanism of the blood pressure measurement device according to Example 2.

### DESCRIPTION OF EMBODIMENTS

### Example 1

Examples of the present invention will be specifically described below with reference to the drawings. Note that the material, shape, relative arrangement, and the like of configurations described in this example are not intended to limit the scope of this invention to the configurations alone, unless otherwise stated.

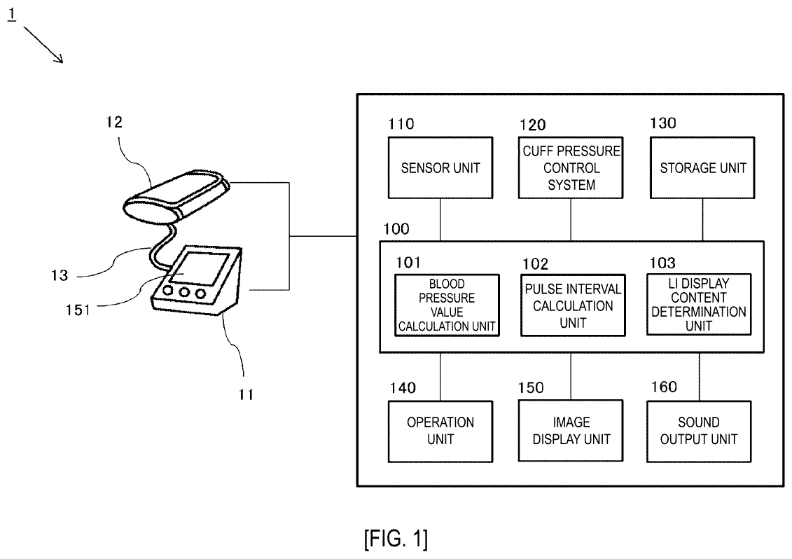

The present invention can be applied to, for example, a blood pressure measurement device 1 as illustrated in FIG. 1. FIG. 1 is a schematic diagram illustrating an outline of a device configuration and a functional configuration of the blood pressure measurement device 1 according to the present example. As illustrated in FIG. 1, the blood pressure measurement device 1 generally includes a main body portion 11, a cuff portion 12, and an air tube 13. As indicated by the functional blocks in FIG. 1, the blood pressure measurement device 1 includes functional units of a control unit 100, a sensor unit 110, a cuff pressure control system 120, a storage unit 130, an operation unit 140, an image display unit 150, and a sound output unit 160.

Although not illustrated, the main body portion 11 includes an image display mechanism 151 such as a liquid crystal display (LCD), various operation buttons, a sound output mechanism such as a speaker, a power supply portion such as a battery, a pump and a valve communicating with the cuff portion, a housing accommodating these components, and the like. The cuff portion 12 is a member used by being wrapped around an upper arm of a user, and includes an air bag (cuff) communicating with the pump and the valve of the main body portion 11 via the air tube 13, a belt incorporating the cuff, a pressure sensor provided at the belt (none of which are illustrated), and the like. When blood pressure measurement is performed by the Korotkoff method, a microphone may be included.

The belt of the cuff portion 12 is provided with a fixing member (for example, a hook-and-loop fastener) for fixing the cuff portion 12 to the upper arm of the user, and the cuff portion 12 is wrapped around the upper arm of the user by the belt when blood pressure measurement is performed using the blood pressure measurement device 1.

The control unit 100 is a unit for controlling the blood pressure measurement device 1, and includes, for example, a central processing unit (CPU). Upon receiving operation by the user via the operation unit 140, the control unit 100 controls each component of the blood pressure measurement device 1 to execute various types of processing such as blood pressure measurement and provision of various types of information in accordance with a predetermined program. The predetermined program is stored in the storage unit 130 described below to be read therefrom. The control unit 100 includes a blood pressure value calculation unit 101, a pulse interval calculation unit 102, and a level indicator display content determination unit 103 as functional modules. These functional modules will be described in detail below.

The sensor unit 110 includes the pressure sensor (for example, a piezoresistive sensor including a piezoelectric element) provided at the cuff portion as described above, and detects at least a pulse wave of the user. The sensor unit 110 may include a sensor other than the pressure sensor, and may include a photoplethysmography (PPG) sensor when a pulse wave is detected by a photoelectric method. The blood pressure measurement device 1 according to the present example acquires a pulse of the user based on a pulse wave detected by the sensor unit 110. That is, in the present example, the sensor unit 110 corresponds to a pulse acquisition mechanism.

The cuff pressure control system 120 controls the pump and the valve of the main body portion 11 to adjust a cuff pressure of the cuff portion 12 at the time of blood pressure measurement. Specifically, at the time of blood pressure measurement, control is performed such that the pump is driven in a state where the cuff portion 12 is wrapped around the upper arm to feed air into the cuff so as to inflate the cuff (increase a cuff pressure). In this way, the blood flow is once blocked by compressing the blood vessel of the upper arm of the user, and then control is performed such that the pump is stopped and the valve is opened to gradually release air from the cuff so as to deflate the cuff (decrease the cuff pressure).

The storage unit 130 includes a main storage device such as a random access memory (RAM), a read only memory (ROM), or the like, and an auxiliary storage device such as a hard disk drive (HDD), a flash memory, or the like, and stores various types of information such as application programs, various type of measurement results such as blood pressure values, pulse waves, and other biological information acquired. Measured blood pressure values, measured pulse waves, and the like may be stored in the storage unit 130 in association with time information such as acquisition times and measurement times. As the time information, for example, information measured with reference to a real time clock (RTC) can be used. The auxiliary storage device may be configured to be attachable to and detachable from the main body portion 11.

The operation unit 140 includes components such as a power button, a measurement execution button, and a selection and determination button, for example, and has a function of receiving input operation from the user and causing the control unit 100 to execute processing in accordance with the operation.

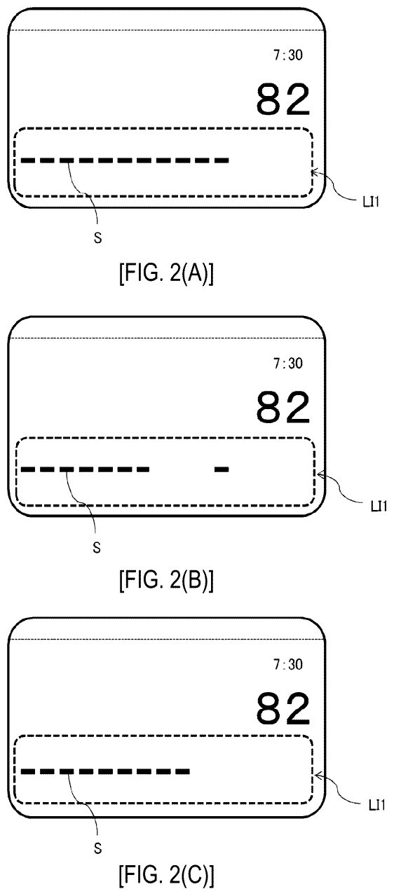

The image display unit 150 includes the image display mechanism 151 of the main body portion 11, and provides the user with information by displaying various types of information such as measured blood pressure values, the current time, and information regarding a cuff attachment state at the image display mechanism 151. FIG. 2(A) to FIG. 2(C) illustrate examples of the display content of the image display mechanism 151. As illustrated in FIG. 2(A) to FIG. 2(C), the image display mechanism 151 is provided with a region for displaying a level indicator LI1 indicating information about a pulse interval described below. The level indicator LII will be described in further detail below.

The sound output unit 160 includes a sound generation portion such as a speaker, and presents information to the user by sound. Specifically, the sound output unit 160 may generate an announcement of blood pressure measurement start or a guide sound related to the use of the device. Information on a pulse interval may be output by sound as described below.

Hereinafter, each functional module of the control unit 100 will be described. The blood pressure value calculation unit 101 calculates a blood pressure value (and a pulse rate) of the user based on a pulse wave acquired by the sensor unit 110. As a blood pressure calculation method, any desired known technique can be used and, for example, an oscillometric method in which a blood pressure is measured by detecting a pressure pulse wave using a pressure sensor can be employed. A microphone may be provided at the cuff portion 12 and the Korotkoff method for detecting Korotkoff sounds may be used. The blood pressure value and the pulse rate calculated by the blood pressure value calculation unit 101 may be stored in the storage unit 130 in association with a blood pressure measurement time.

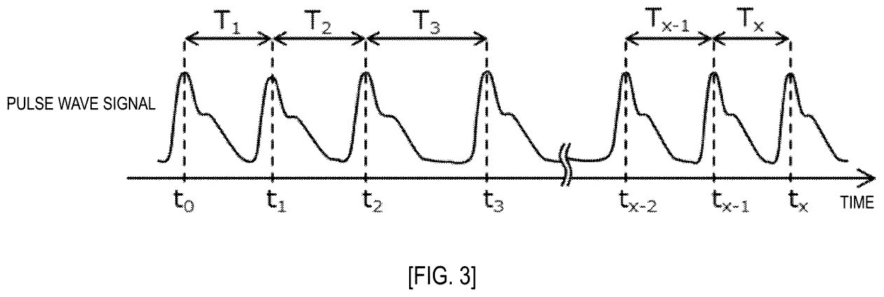

The pulse interval calculation unit 102 calculates, from a waveform of the pulse wave acquired by the sensor unit 110 (for example, a pressure pulse wave acquired by the pressure sensor), an inter-peak time interval of the pulse wave for each beat. The calculation of the pulse interval will be described with reference to FIG. 3. FIG. 3 is an explanatory diagram schematically illustrating a relationship between a pulse wave signal and a time. In FIG. 3, when the time at which the peak of a certain wave is detected is defined as t₀ and the time at which the peak of the next wave is detected is defined as t₁, the interval between the certain wave and the next wave is t₁−t₀=T₁. In this way, the pulse interval calculation unit 102 calculates tₓ−tₓ₋₁=Tₓ as the pulse interval.

The level indicator display content determination unit 103 determines the display content of the level indicator LI1 to be displayed at the image display mechanism 151. Here, the display content of the level indicator LI1 will be described with reference to FIG. 2(A) to FIG. 2(C). The level indicator LI1 according to the present example includes a plurality of display segments S capable of being switched between a display state and a non-display state, and can indicate the magnitude of the calculated pulse interval by the number of display segments S in a state in which the display is activated (in a display state, not in a non-display state). The level indicator display content determination unit 103 then determines the number of display segments S to be activated based on the pulse interval Tₓ calculated for each beat, and determines the display content in the level indicator LI1 for each beat.

Here, a specific example in which the level indicator display content determination unit 103 determines the number of display segments S to be activated will be described. The level indicator display content determination unit 103 obtains the number Nₓ of display segments S corresponding to the calculated pulse interval by Equations (1) and (2) described below, where Tₘᵢₙ is a predetermined minimum value of the pulse interval, Tₘₐₓ is a predetermined maximum value of the pulse interval, and Nₘₐₓ is the maximum number of display segments S (these values are set in advance).

$$\begin{matrix}[ Equation 1 ] & \\ Δ t = ( {T}_{max} - {T}_{min} ) / ( {N}_{max} - 1 ) & ( 1 )\end{matrix}$$

$$\begin{matrix}[ Equation 2 ] & \\ {N}_{x} = [ ( {T}_{x} - {T}_{min} ) / Δ t + 1 ] & ( 2 )\end{matrix}$$

That is, Nₓ is the maximum integer not exceeding the value of (Tₓ−Tₘᵢₙ)/Δt+1. Δt does not need to be obtained each time, and a value predetermined for each device or each user may be stored in the storage unit 130. Alternatively, only Equation (2) in which the value of Δt is determined may be stored in the storage unit 130, and Nₓ may be obtained by Equation (2) for each beat.

By obtaining Nₓ in this way and displaying Nₓ display segments S at the level indicator LI for each beat, it is possible to indicate a fluctuation in the pulse interval. Specifically, when the number of display segments S in which the display is activated is large, the display activation region of the level indicator LI becomes large, and conversely, when the number of display segments S in which the display is activated is small, the display activation region of the level indicator LI becomes small. That is, the display activation region of the level indicator LI becomes larger as the pulse interval becomes larger (longer), and the display activation region of the level indicator LI becomes smaller as the pulse interval becomes smaller (shorter). Thus, the user can intuitively recognize a variation in the size of the display activation region by viewing the display, thereby recognizing an amount of change of the pulse interval for each beat.

Contriving a display mode enables the display segments S displayed on the level indicator LI to indicate the amount of change in the pulse interval to the user in an easy-to-understand manner. For example, in a mode in which the display segments S are arranged in a straight line extending in the left-right direction as illustrated in FIG. 2(A) to FIG. 2(C), the display activation of the display segments S starts from the leftmost display segment S, the number of segments S to be displayed is sequentially increased rightward one by one until Nₓ display segments S are displayed, and after the Nₓ display segments S are displayed, the display segments S are sequentially hidden from the right.

FIG. 2(A) to FIG. 2(C) illustrate an example of such a display transition of the display segments S. FIG. 2(A) illustrates the level indicator LI1 in a case where Nₓ is 10 as an indication of the pulse interval Tₓ at a certain point in time. In this example, Nₘₐₓ is 13. That is, a state is illustrated in which there are 13 display segments S in the level indicator LI1 as a whole, and 10 display segments S are displayed (activated) in a left-aligned manner.

FIG. 2(B) illustrates a state in which the display segments S are sequentially hidden from the right. As illustrated in FIG. 2(B), the display of the Nₓ-th display segment S (in this case, the tenth display segment S) remains activated when the display segments S are sequentially hidden, and thus it is possible to make it easy to recognize a fluctuation in the pulse interval from a change in the position of the remaining display segment S. However, this is not necessarily required, and the display of all the Nₓ display segments S may be simultaneously activated and all the Nₓ display segments S may remain displayed until the next pulse interval is obtained.

When the peak of the next pulse wave is detected and the level indicator display content determination unit 103 determines the display content of the pulse interval Tₓ₊₁ calculated based thereon, eight display segments S are displayed in an activated state in the level indicator LI1 as illustrated in FIG. 2(C). In the present example, the display of the level indicator LI1 changes in synchronization with the waveform of the detected pulse. That is, when the pulse interval is short, the timing at which the display of the level indicator LI1 changes becomes fast accordingly, and when the pulse interval is long, the timing at which the display changes becomes slow. However, the timing at which the display of the level indicator LI1 changes does not necessarily need to be synchronized with the pulse wave, and it is possible to appropriately set timings at which the display indicating the pulse interval starts and ends for each beat.

In addition to the change in the display of the level indicator LI1 as described above, a sound indicating the pulse interval (for example, an electronic sound such as “beep, beep, . . . ”) may be output from the sound output unit 160. For example, specifically, when the pulse interval Tₓ at a certain point in time is longer than a predetermined reference, an electronic sound having a low frequency is output, and conversely, when the pulse interval Tₓ is shorter than the predetermined reference, an electronic sound having a high frequency is output. When the pulse interval Tₓ has a length within a predetermined range defined by upper and lower limit threshold values, an electronic sound in a frequency band between the above frequencies may be output.

According to the blood pressure measurement device 1 having the above-described configuration, the user can intuitively recognize a fluctuation in the pulse interval during blood pressure measurement and can more easily recognize the beat of the pulse. Thus, when there is an abnormality such as arrhythmia, a pulse abnormality can be easily recognized from a sense of discomfort thereof, which can contribute to early detection of a cardiovascular disease through daily blood pressure measurement.

### Modified Example 1

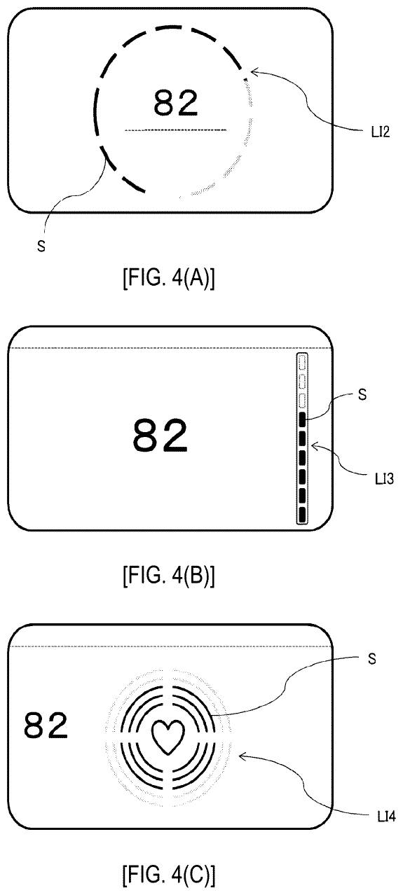

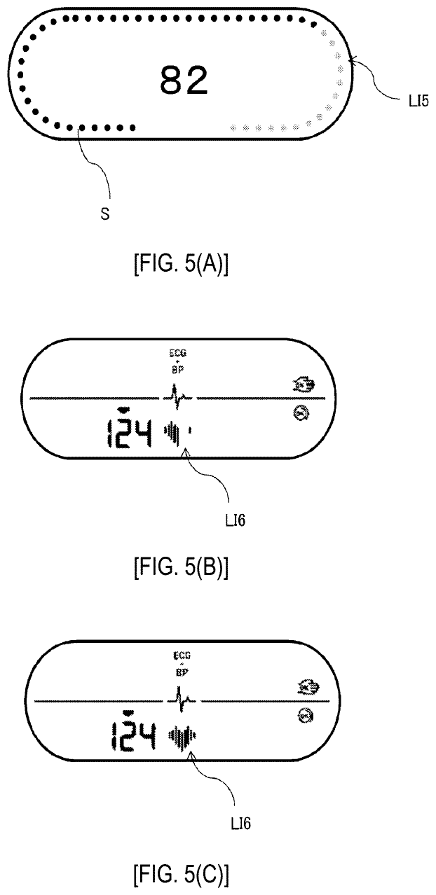

In the above-described example, the level indicator LI1 is configured such that the display segments S are arranged in a straight line extending in the left-right direction, but the level indicator can be configured in various ways in accordance with the shape of the device and the structure of the image display mechanism 151. FIG. 4(A) to FIG. 4(C) and FIG. 5(A) to FIG. 5(C) illustrate display modes of level indicators according to a modified example of Example 1.

The level indicator can have a circular shape, not a linear shape, like a level indicator LI2 illustrated in FIG. 4(A). Like a level indicator LI3 illustrated in FIG. 4(B), the level indicator may be configured in a linear shape extending in the vertical direction. Further, the level indicator can be a level indicator LI4 having a mode in which display segments S radially arranged in a plurality of lines, and the display is activated from the inside toward the outside, as illustrated in FIG. 4(C). The level indicator can be a level indicator LI5 in which each display segment S is formed not in a linear shape but in a dot shape, as illustrated in FIG. 5(A). As illustrated in FIG. 5(B) and FIG. 5(C), a display region of a level indicator LI6 can be designed so as to have a certain figure (for example, a heart shape) when all display segments S constituting the level indicator LI6 are activated. FIG. 5(B) illustrates a state in which the display of some display segments of the level indicator LI6 having a heart-shaped display region is activated. FIG. 5(C) illustrates a state in which the display of all the display segments is activated.

### Modified Example 2

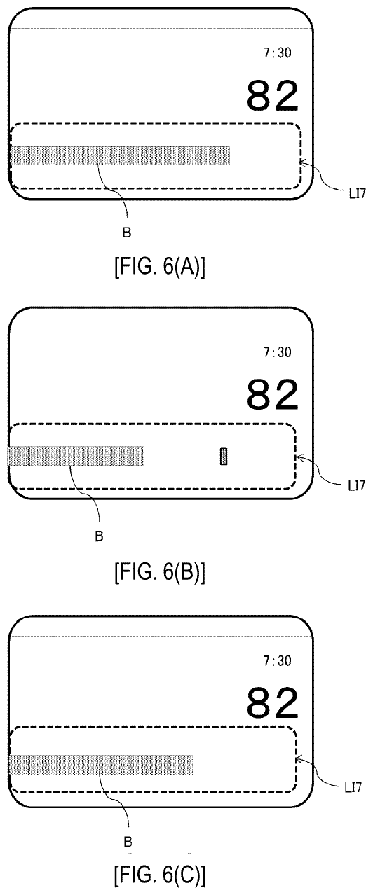

The level indicator does not necessarily need to be composed of a plurality of display segments. FIG. 6(A) to FIG. 6(C) illustrate examples of a level indicator LI7 in such a case. FIG. 6(A) illustrates a state in which a continuous bar B extends from the left to the right as an indication of the pulse interval Tₓ at a certain point in time. In the case of this modified example, Nₓ may be calculated not as the number of display segments but as the length (or area) of a display activation region in an entire displayable region of the level indicator LI7. FIG. 6(B) illustrates a state in which the bar is sequentially hidden from the right. At this time, as in Example 1, the display of a peak level portion of the bar B in FIG. 6(A) remains activated. FIG. 6(C) illustrates a state in which the bar indicating the pulse interval extends from the left to the right and a pulse interval Tₓ₊₁ is displayed.

### Modified Example 3

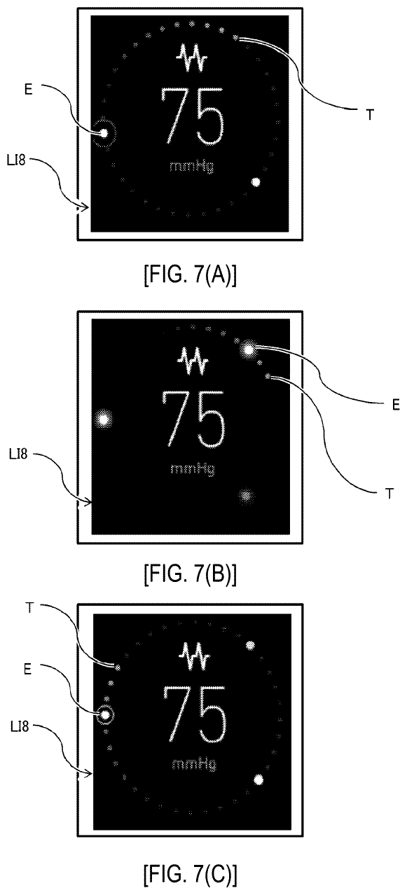

In each of the examples described above, the pulse interval is represented by the size (including the length) of the display activation region in the level indicator, but the pulse interval may be displayed in another mode. Specifically, for example, a display segment in which the display is activated may sequentially transition, and the pulse interval may be represented by the length of the distance of the transition of the activated display segment. FIG. 7(A) to FIG. 7(C) are explanatory diagrams illustrating a level indicator LI8 in such a modified example.

In the examples of the level indicator LI8 illustrated in FIG. 7(A) to FIG. 7(C), a plurality of display segments are arranged in a ring shape, and, in this mode, a display segment sequentially activated transitions in a clockwise direction during blood pressure measurement. In each of FIG. 7(A) to FIG. 7(C), a display segment located at a leading end of the transition is denoted as a leading display segment T, and a display segment indicating an end point of the transition of one pulse interval is denoted as a terminal display segment E.

The transition of the display segment will be further described. When the pulse interval Tₓ is calculated by the pulse interval calculation unit 102, the level indicator display content determination unit 103 determines how many display segments an advance (transition) is to be made based on the calculated pulse interval T. When the transition is made by the determined number of display segments and the terminal display segment E of the transition indicating one pulse interval is activated, the terminal display segment E is displayed in a special manner different from those of passing points of the transition. Specifically, for example, the terminal display segment E may be displayed slightly larger, the brightness of the terminal display segment E may be higher than those of normal transitions, a display effect may be added as illustrated in FIG. 7(C), or a combination thereof may be performed. By performing such display, it is possible to easily recognize that this segment is the end of the transition indicating one pulse interval.

The display of display segments once activated, including passing points of the transition, are not deactivated immediately after the activated display segment transitions to the next one, but the brightness of the display may be set to gradually decrease toward a non-display state. As a result, as illustrated in FIG. 7(A) to FIG. 7(C), it is possible to give an impression that the leading display segment T at the leading end of the transition circles while leaving an afterimage. Furthermore, the display of the terminal display segment E may be activated for a longer time than those of the display segments at the passing points. Accordingly, the previous pulse interval and the next pulse interval can be easily compared with each other, and the user can more clearly recognize a fluctuation in the pulse interval.

### Modified Example 4

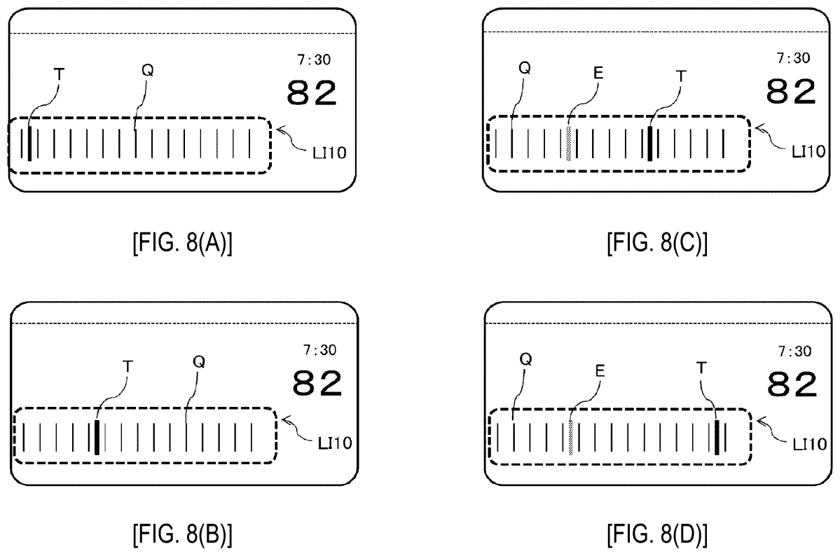

The mode in which the activated display segment transitions in a fixed direction as illustrated in Modified Example 3 can be applied to other than a level indicator formed in a ring shape. FIG. 8(A) to FIG. 8(D) illustrate modified examples of such a case. In the present modified example, a mode is adopted in which a display segment sequentially activated in one direction transitions from the left end to the right end of a level indicator LI10 extending in the left-right direction during blood pressure measurement. In FIG. 8(A) to FIG. 8(D) as well, a display segment located at a leading end of the transition is indicated as a leading display segment T, and in FIG. 8(C) and FIG. 8(D), a display segment indicating a terminal end of the transition of one pulse interval is indicated as a terminal display segment E.

As illustrated in FIG. 8(A) to FIG. 8(D), the level indicator LI10 according to the present modified example includes a plurality of display segments in a display region extending in the left-right direction, and the display segments are partitioned by a plurality of partition lines Q that are always displayed. In the present modified example as well, how many display segments a transition is to be made is determined based on a calculated pulse interval as in Modified Example 3. The transition is then made by the determined number of display segments and the terminal display segment E of the transition indicating one pulse interval is activated (see FIG. 8(A) and FIG. 8(B)). Thereafter, the leading display segment T is sequentially activated toward the right end of the level indicator LI10, while the terminal display segment E is kept activated (see FIG. 8(C) and FIG. 8(D)). When the transition of the leading display segment T reaches the right end of the level indicator LI10, the display segment is sequentially activated again from the left end of the level indicator LI10.

In the present modified example, even after the terminal display segment E is displayed, the leading display segment T transitions toward the right end of the level indicator LI10. However, when the display of the terminal display segment E is activated, the transition of the leading display segment T toward the right end may be stopped and the display may be activated again from the left end. In the present modified example, the level indicator LI10 is formed in a band shape extending in the left-right direction, but a level indicator extending in the up-down direction can naturally have a display mode similar to that of the present modified example.

### Modified Example 5

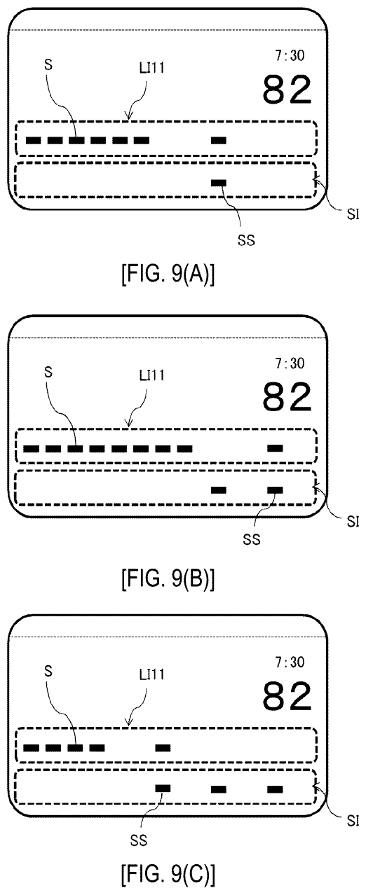

Although the level indicator including the plurality of display segments S is displayed in Example 1, another indicator in addition to this level indicator may be displayed at the image display unit 150. FIG. 9(A) to FIG. 9(C) illustrate modified examples of such a case. In the present modified example, as illustrated in FIG. 9(A) to FIG. 9(C), a level indicator LI11 having a configuration similar to that of the level indicator LI1 of Example 1 and a sub-indicator SI disposed below and in parallel with the level indicator LI11 are displayed at the image display portion.

As illustrated in FIG. 9(A) to FIG. 9(C), the sub-indicator SI includes a plurality of sub-display segments SS. As in Example 1 illustrated in FIG. 2(A) to FIG. 2(C), display segments S in the level indicator LI11 are activated from the left toward the right for each beat. After a number of display segments S corresponding to a pulse interval are activated, the display segment S of the peak level is kept activated, and the left display segments thereof are sequentially hidden. Here, in the sub-indicator SI, the display of a sub-display segment SS at a position corresponding to the position of the display segment S of the peak level of the level indicator LI11 in the left-right direction is activated (see FIG. 9(A)). In the sub-indicator SI, only the sub-display segment SS at the position corresponding to the display segment S of the peak level is activated, unlike the level indicator LI11.

Thereafter, in the level indicator LI11, the display segments S indicating the pulse interval related to the next beat are activated, and the position of the display segment S indicating the peak level changes accordingly. That is, the display segment S of the peak level indicating the pulse interval of the previous beat is hidden. On the other hand, in the sub-indicator SI, while the display of the sub-display segment SS at the position corresponding to the peak level of the previous beat is kept activated, the display of a sub-display segment SS at a position corresponding to the position of the peak level of the next beat is also activated (see FIG. 9(B) and FIG. 9(C)). The activation of the display of the respective sub-display segments SS of the sub-indicator SI is maintained until blood pressure measurement is finished (that is, until acquisition of the pulse is finished).

In such a display mode, it is possible to easily recognize the degree of variation in the pulse interval. That is, at the end of blood pressure measurement, the larger the number of activated sub-display segments SS in the sub-indicator SI is and the wider the range of the displayed positions is, the larger the variation in the pulse interval is. When atrial fibrillation occurs at the time of blood pressure measurement, a variation in the pulse interval becomes large, and thus the user can sensuously recognize whether or not there is a risk of atrial fibrillation by checking a mode of display activation of the sub-display segments SS of the sub-indicator SI.

### Example 2

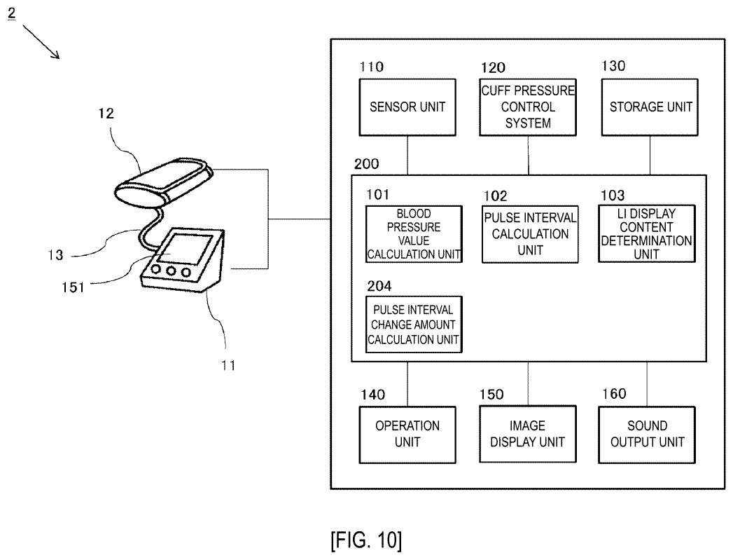

Next, Example 2 of the present invention will be described. FIG. 10 is a schematic diagram illustrating an outline of a device configuration and a functional configuration of a blood pressure measurement device 2 according to the present example. As illustrated in FIG. 10, the blood pressure measurement device 2 has substantially the same configuration as that of the blood pressure measurement device 1 of Example 1 except that the blood pressure measurement device 2 includes a pulse interval change amount calculation unit 204 as a functional module of a control unit 200. Thus, the same components and the same functional modules as those of the blood pressure measurement device 1 are denoted by the same reference numerals as those of Example 1, and detailed description thereof will be omitted.

The pulse interval change amount calculation unit 204 calculates, based on a pulse interval calculated by a pulse interval calculation unit 102, an amount of change of the pulse interval. The amount of change may be a difference or a ratio between the latest pulse interval and the pulse interval calculated immediately before.

Referring to FIG. 3, for example, when a pulse interval difference Dₓ is obtained, the pulse interval difference Dₓ can be calculated by Equation (3) described below. That is, when a certain pulse interval Tₓ is calculated, a pulse interval Tₓ₋₁ calculated immediately before is subtracted from the pulse interval Tₓ, and thus the difference (pulse interval difference Dₓ) between the latest pulse interval Tₓ and the immediately preceding pulse interval Tₓ₋₁ is calculated.

$$\begin{matrix}[ Equation 3 ] & \\ {D}_{x} = {T}_{x} - {T}_{x - 1} & ( 3 )\end{matrix}$$

When a pulse interval ratio Rₓ is obtained, the pulse interval ratio Rₓ can be calculated by Equation (4) described below. That is, when a certain pulse interval Tₓ is calculated, the pulse interval Tₓ is divided by a pulse interval Tₓ₋₁ calculated immediately before, and thus the ratio (pulse interval ratio Rₓ) between the latest pulse interval Tₓ and the immediately preceding pulse interval Tₓ₋₁ is calculated.

$$\begin{matrix}[ Equation 4 ] & \\ {R}_{x} = {T}_{x} / {T}_{x - 1} & ( 4 )\end{matrix}$$

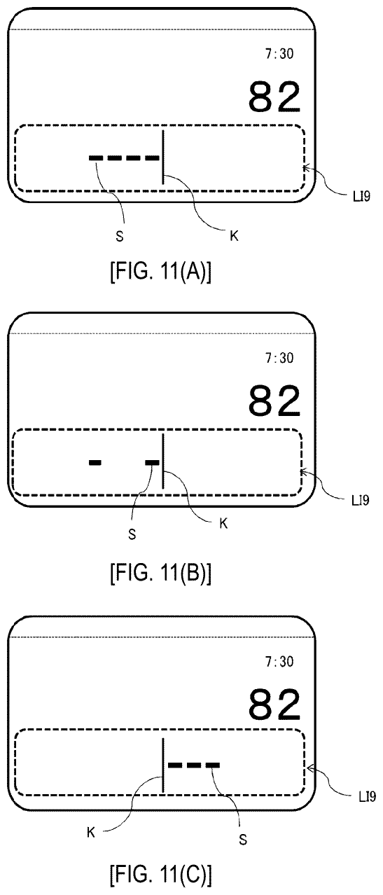

A level indicator display content determination unit 103 then determines a display content indicating Dₓ or Rₓ calculated as described above. Here, a display content of a level indicator LI9 displayed at an image display mechanism 151 in the present example will be described with reference to FIG. 11(A) to FIG. 11(C). The level indicator LI9 in the present example is in a state in which a vertical reference line K is always displayed at the center. Display segments S are arranged so as to extend in the left-right direction across the vertical reference line K.

When indicating Dₓ with, for example, the level indicator LI9, the level indicator display content determination unit 103 determines the display content so as to display a number of display segments S corresponding to |Dₓ| on the right side of the reference line K when Dₓ>0 and on the left side of the reference line K when Dₓ<0. When Rₓ is indicated by the level indicator LI9, a number of display segments S corresponding to |Rₓ| can be similarly displayed on the right side of the reference line K when Rₓ>1 and on the left side of the reference line K when Rₓ<1.

FIG. 11(A) to FIG. 11(C) illustrate an example of such a display transition of the display segments S. FIG. 11(A) illustrates the level indicator LI9 in a state in which four display segments S are displayed on the left side of the reference line K as an indication of the difference Dₓ between the pulse interval Tₓ at a certain point in time and the immediately preceding pulse interval Tₓ₋₁ (i.e., a variation of the pulse interval). That is, it can be seen that Dₓ at this point in time is a negative value, and the pulse interval Tₓ is shorter than the immediately preceding pulse interval Tₓ₋₁.

FIG. 11(B) illustrates a state in which the display segments S are sequentially hidden from the left end. As illustrated in the FIG. 11(B), the display of the leftmost display segment S indicating the immediately preceding Dₓ remains activated when the display segments are sequentially hidden, which can make it easy to recognize a fluctuation in the amount of change of the pulse interval from a change of the position of the remaining display segment S. FIG. 11(C) illustrates the level indicator LI9 in a state in which the difference (Dₓ₊₁=Tₓ₊₁−Tₓ) between the pulse interval Tₓ and the next pulse interval (Tₓ₊₁) is displayed. Here, three display segments S are displayed on the right side of the reference line K, and it is possible to easily recognize that the pulse interval Tₓ₊₁ is longer than the pulse interval Tₓ and the degree thereof.

According to the blood pressure measurement device 2 of the present example, the user can easily and intuitively recognize the fluctuation in the amount of change in the pulse interval and the degree thereof for each per visually recognizing the direction in which the display segments S of the level indicator LI9 are activated and the length thereof.

### Other Points

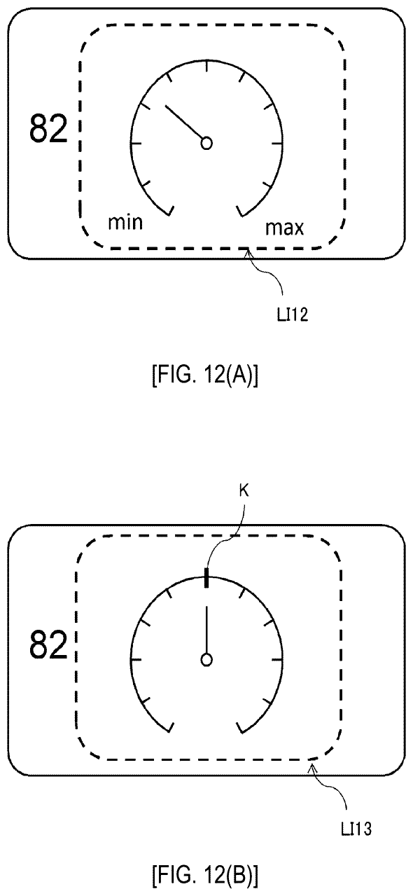

The description of the examples described above is merely illustrative of the present invention, and the present invention is not limited to the specific examples described above. Within the scope of the technical idea of the present invention, various modifications and combinations may be made. For example, the method of representing the amount of change in the pulse interval and the fluctuation thereof is not limited to an increase or decrease of the display region (the number of display segments in which the display is activated, the length or the area of the bar) in the level indicator as described above. FIG. 12(A) and FIG. 12(B) illustrate display modes of such level indicators. The level indicators illustrated in FIG. 12(A) and FIG. 12(B) have a shape including a part of a circumference (arc) and a pointer extending from the inside of the arc toward the arc, and have a configuration like a so-called analog meter.

FIG. 12(A) is a diagram illustrating a modified example of Example 1 for indicating the pulse interval for each beat. A pointer of a level indicator LI12 is displayed so as to indicate any position between min and max on a circumference corresponding to the pulse interval for each beat. That is, the angle of the pointer changes for each beat in accordance with the pulse interval.

On the other hand, FIG. 12(B) is a diagram illustrating a modified example of Example 2 for indicating an amount of change in the pulse interval for each beat. A pointer of a level indicator LI13 is displayed so as to indicate a position on an arc corresponding to the difference or ratio between the latest pulse interval and the immediately preceding pulse interval for each beat. Specifically, the level indicator LI13 is displayed such that the pointer swings to the right or left from a reference line K in accordance with the amount of change in the pulse interval with the reference line K disposed at the center of the arc as a reference position. That is, according to the description of Example 2, when Dₓ>0, the latest pulse interval is longer than the immediately preceding pulse interval, so that the pointer swings to the right of the reference line K by an amount corresponding to |Dₓ|. When Dₓ<0, the latest pulse interval is shorter than the immediately preceding pulse interval, so that the pointer swings to the left of the reference line K by an amount corresponding to |Dₓ|. The same also applies to the case of indicating the ratio (Rₓ described above) between the latest pulse interval and the immediately preceding pulse interval.

In each of the above-described examples, the length, the area, or the angle of the region in which the display is activated or the number of the regions in the level indicator can linearly change in accordance with the pulse interval or the amount of change thereof. Since the region or regions in which the display is activated in the level indicator change in proportion to the pulse interval or the amount of change thereof in this way, the user can intuitively and visually recognize the fluctuation in the pulse interval or the amount of change in the pulse interval. However, the size of the display region of the device is limited, and thus when the region in which the display is activated changes in proportion to the pulse interval in this way, there is a concern that the fluctuation in the pulse interval may be difficult to understand in accordance with the magnitude of the pulse rate of the user.

For this reason, the length, the area, the angle of the region in which the display is activated or the number of the regions in the level indicator may change in a monotonically increasing non-linear manner in accordance with the pulse interval or the amount of change thereof. Specifically, for example, calculation processing may be performed for each beat using the calculated pulse interval, and the number of segments in which the display is activated may change in proportion to the logarithm of the pulse interval. According to this, it is possible to suppress the variation due to the magnitude of the pulse rate and to indicate the pulse interval or the amount of change thereof by appropriately changing the display. A table in which the calculated pulse interval or the amount of change thereof is associated with the number of segments in which the display is to be activated may be stored in advance, and the display of a number of segments corresponding to the table may be activated with reference to the table for each beat. According to this, it is possible to reduce the load of performing the calculation processing for each beat.

Although the level indicator is displayed at the LCD in each of the above-described examples, the display segments may be composed of a plurality of LED indicator lights. In such a case, turning on the LED indicator lights corresponds to the activation of the display segments.

Although the pressure pulse wave is acquired by the pressure sensor in each of the above-described examples, a volume pulse wave may be acquired by a PPG sensor. Although the blood pressure measurement device has been described as an example in each of the above examples, the present invention is not limited thereto and may be applied to other biological information measurement devices (for example, an electrocardiograph, a body composition meter, and the like) as long as the devices include a sensor capable of acquiring a pulse.

### REFERENCE NUMERALS LIST

1, 2 Blood pressure measurement device
11 Main body portion
12 Cuff portion
13 Air tube
151 Image display mechanism
100, 200 Control unit
110 Sensor unit
120 Cuff pressure control system
130 Storage unit
140 Operation unit
150 Image display unit
160 Sound output unit
LI1, LI2, LI3, LI4, LI5, LI6, LI7, LI8, LI9, LI10, LI11, LI12, LI13 Level indicator
SI Sub-indicator
S Display segment
SS Sub-display segment
T Leading display segment
E Terminal display segment
B Bar
K Reference line

1. A biological information measurement device, comprising:
a pulse acquisition mechanism configured to detect a pulse of a human body;
a pulse interval calculation mechanism configured to calculate, based on the pulse, a pulse interval between one beat and an immediately preceding beat; and
a display mechanism configured to display a level indicator visually indicating the pulse interval, wherein
the level indicator indicates, per beat of the pulse detected, the pulse interval.

2. The biological information measurement device according to claim 1, wherein
the level indicator visually indicates the pulse interval by using at least one of a length, an area, or an angle of a region in which display is activated at the display mechanism or the number of the regions.

3. The biological information measurement device according to claim 2, wherein
the level indicator visually indicates the pulse interval by using a size indicated by a display region in which display is activated in the level indicator.

4. The biological information measurement device according to claim 3, wherein
the level indicator is composed of a plurality of display segments and represents the size of the display region depending on whether the number of the plurality of display segments in which display is activated is large or small.

5. The biological information measurement device according to claim 3, wherein
the level indicator maintains activation of display of a peak level portion of one pulse interval from display of the one pulse interval until display of a next pulse interval.

6. A biological information measurement device, comprising:
a pulse acquisition mechanism configured to detect a pulse of a human body;
a pulse interval calculation mechanism configured to calculate, based on the pulse, a pulse interval between one beat and an immediately preceding beat; and
a display mechanism configured to display a level indicator visually indicating at least one of the pulse interval or an amount of change from another pulse interval immediately before the pulse interval, wherein
the level indicator is composed of a plurality of display segments and visually indicates, per beat of the pulse, the pulse interval or the amount of change depending on whether the number of the plurality of display segments in which display is activated is large or small,
the display mechanism further displays a sub-indicator including a plurality of sub-display segments corresponding one-to-one to the plurality of display segments of the level indicator,
the level indicator maintains activation of display of a peak level portion of one pulse interval or one amount of change from display of the one pulse interval or the one amount of change until display of a next pulse interval or a next amount of change, and
the sub-indicator activates, per beat, display of a sub-display segment of the plurality of sub-display segments that corresponds to the display of the peak level portion of the pulse interval or the amount of change indicated per beat by the level indicator and maintains display of each sub-display segment activated until the detection by the pulse acquisition mechanism is finished.

7. A biological information measurement device, comprising:
a pulse acquisition mechanism configured to detect a pulse of a human body;
a pulse interval calculation mechanism configured to calculate, based on the pulse, a pulse interval between one beat and an immediately preceding beat; and
a display mechanism configured to display a level indicator visually indicating at least one of the pulse interval or an amount of change from another pulse interval immediately before the pulse interval, wherein
the level indicator is composed of a plurality of display segments,
a display segment in which display is activated in the level indicator makes a transition, and
the pulse interval or the amount of change is visually indicated per beat of the pulse by a length of a distance of the transition.

8. The biological information measurement device according to claim 7, wherein
the level indicator represents, per transition of the display segment indicating one pulse interval or one amount of change, a length of a distance of the transition by highlighting the display segment activated at a terminal end of the distance of the transition.

9. The biological information measurement device according to claim 8, wherein
the level indicator maintains, until display of the display segment indicating the terminal end of the transition of the display segment indicating the one pulse interval or the one amount of change is activated, activation of the display segment indicating the terminal end of the transition related to at least an immediately preceding pulse interval or an immediately preceding amount of change.

10. The biological information measurement device according to claim 7, wherein
the level indicator has an entire display region configured in a ring shape,
the display segment in which the display is activated in the level indicator repeats, per beat, a transition in a fixed direction, and
the pulse interval is indicated by the length of the distance of the transition.

11. The biological information measurement device according to claim 7, wherein
the level indicator has an entire display region configured in a band shape extending in a left-right direction,
the display segment in which the display is activated in the level indicator repeats, per beat, a transition in a fixed direction of the left-right direction, and
the pulse interval is indicated by the length of the distance of the transition.

12. The biological information measurement device according to any one of claim 7, wherein
the level indicator has an entire display region configured in a band shape extending in an up-down direction,
the display segment in which the display is activated in the level indicator repeats, per beat, a transition in a fixed direction of the up-down direction, and
the pulse interval is indicated by the length of the distance of the transition.

13. The biological information measurement device according to claim 1, wherein
the level indicator has an entire display region configured in a shape including at least part of a circumference and a pointer extending from an inside of the circumference toward the circumference and visually indicates the pulse interval by using a position on the circumference, the position being indicated by the pointer.

14. The biological information measurement device according to claim 1, wherein
the level indicator indicates the pulse interval in synchronization with a waveform of the pulse detected.

15. The biological information measurement device according to claim 2, wherein
the length, the area, or the angle of the region in which the display is activated or the number of the regions in the level indicator linearly changes in accordance with the pulse interval.

16. The biological information measurement device according to claim 2, wherein
the length, the area, or the angle of the region in which the display is activated or the number of the regions in the level indicator changes in a monotonically increasing non-linear manner in accordance with the pulse interval.

17. The biological information measurement device according to claim 1, further comprising:
a sound output mechanism configured to output, per beat of the pulse detected, a sound indicating the pulse interval in synchronization with display of the level indicator.

18. The biological information measurement device according to claim 17, wherein
the sound output mechanism indicates a difference in the pulse interval depending on a difference in a pitch of an output sound.
